(19) United States
(12) Patent Application Publication
(10) Pub. No.: US 2015/0005502 A1
(43) Pub. Date: Jan. 1, 2015

(54) PROCESS FOR PREPARING [3-[(6-CHLORO-3-PYRIDINYL)METHYL]-2-THIAZOLIDINYLIDENE]CYANAMIDE

(71) Applicant: Bayer Intellectual Property GmbH, Monheim (DE)

(72) Inventors: Norbert Lui, Odenthal (DE); Stefan Antons, Leverkusen (DE); Jens-Dietmar Heinrich, Leverkusen (DE)

(21) Appl. No.: 14/241,538

(22) PCT Filed: Aug. 27, 2012
(86) PCT No.: PCT/EP2012/066602

§ 371 Date: Apr. 28, 2014

(30) Foreign Application Priority Data
Sep. 2, 2011 (EP) 11179883.1

(51) Int. Cl. C07D 417/06

(52) U.S. Cl. CPC C07D 417/06
USPC 546/270.7

(57) ABSTRACT

Process for preparing [3-[(6-chloro-3-pyridinyl)methyl]-2-thiazolidinylidene]cyanamide, comprising the following steps:
(i) reaction of dimethyl N-cyanocarbonimidodithiocarbonate and 2-aminoethanethiol or a salt thereof in the presence of a base;
(ii) reaction of the reaction mixture with 5-chloromethyl-2-chloropyridine,
   which does not need any purification of the cyanimino-1,3-thiazolidine intermediate.

The present invention relates to a process for preparing [3-[(6-chloro-3-pyridinyl)methyl]-2-thiazolidinylidene]cyanamide of the formula (IV), a crop protection active ingredient.

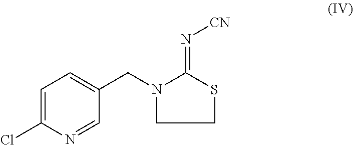 [SMILES: N#C/N=C1\SCCN1Cc1ccc(Cl)nc1]

The preparation is effected in several isolated steps, proceeding from cyanimino-1,3-thiazolidine and 5-chloromethyl-2-chloropyridine (EP 1 024 140), N-(2-chloro-5-pyridylmethyl)cysteamine and dimethyl cyanodithioimidocarbonate (EP 0 235 725). In these cases, the cyanimino-1,3-thiazolidine or the N-(2-chloro-5-pyridylmethyl)cysteamine first have to be prepared and purified, and the end product may also have to be purified. In addition, the overall yield over all stages proceeding from dimethyl N-cyanocarbonimidodithiocarbonate and cysteamine is unsatisfactory.

With regard to the disadvantages and problems outlined above, there is a need to provide a process which, proceeding from dimethyl N-cyanocarbonimidodithiocarbonate, provides [3-[(6-chloro-3-pyridinyl)methyl]-2-thiazolidinylidene]cyanamide without isolation of intermediates, with high yields and high selectivity.

This object is achieved by the following process:

cysteamine or a salt thereof (formula (I))

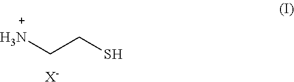

and dimethyl N-cyanocarbonimidodithiocarbonate of the formula (II)

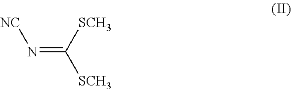

are reacted in the presence of water and optionally with addition of a solvent and of a base, and, without intermediate isolation of cyanimino-1,3-thiazolidine, alkylated with 2-chloro-5-chloromethylpyridine of the formula (III)

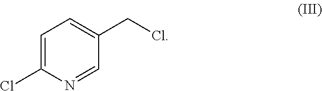

X here is an acid radical, for example halogen, acetate, sulphate or hydrogensulphate.

The reaction proceeds according to Scheme 1:

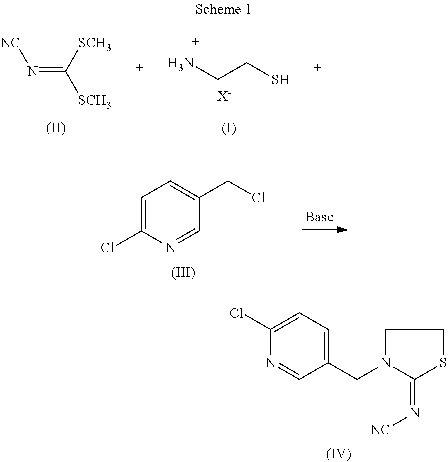

It is surprising that, without intermediate isolation of the cyanimino-1,3-thiazolidine, a higher yield is achieved than when the intermediate is isolated, without any deterioration in the very good quality of the target product.

The application accordingly relates to a process for preparing [3-[(6-chloro-3-pyridinyl)methyl]-2-thiazolidinylidene]cyanamide, comprising the following steps:
(i) reaction of dimethyl N-cyanocarbonimidodithiocarbonate and cysteamine or a salt thereof in the presence of a base;
(ii) reaction of the reaction mixture with 5-chloromethyl-2-chloropyridine.

Preference is given to the performance of the process according to the invention using compounds of the formula (I) in which X is an acid radical, for example halogen, acetate, sulphate or hydrogensulphate.

X is preferably chloride, sulphate or hydrogensulphate.

For the process according to the invention, the bases used are alkali metal and alkaline earth metal hydroxides, alkali metal carbonates or alkali metal hydrogencarbonates. Preference is given to using sodium hydroxide, potassium hydroxide, sodium hydrogencarbonate, potassium hydrogencarbonate, sodium carbonate and potassium carbonate.

The cysteamine salts of the formula (I) for use as starting materials in the process according to the invention are commercially available and commonly known compounds in organic chemistry.

The process according to the invention proceeds in the presence of water. It is also possible to use water-containing solvent mixtures. As well as water, these may also contain other solvents. Examples include alcohols such as methanol, ethanol, isopropanol, n-butanol, isobutanol; ethers such as ethyl propyl ether, methyl tert-butyl ether, n-butyl ether, anisole, phenetole, cyclohexyl methyl ether, dimethyl ether, diethyl ether, dimethylglycol, diphenyl ether, dipropyl ether, diisopropyl ether, di-n-butyl ether, diisobutyl ether, diisoamyl ether, ethylene glycol dimethyl ether, isopropyl ethyl ether, tetrahydrofuran, dioxane, dichlorodiethyl ether and polyethers of ethylene oxide and/or propylene oxide; amines such as trimethyl-, triethyl-, tripropyl-, tributylamine, N-methylmorpholine, pyridine and alkylated pyridines; nitriles such as acetonitrile, propionitrile, butyronitrile, isobutyronitrile, benzonitrile, phenyl nitrile, m-chlorobenzonitrile and compounds such as tetrahydrothiophene dioxide and dimethyl sulphoxide, tetramethylene sulphoxide, dipropyl sulphoxide, benzyl methyl sulphoxide, diisobutyl sulphoxide, dibutyl sulphoxide, diisoamyl sulphoxide; sulphones such as dimethyl, diethyl, dipropyl, dibutyl, diphenyl, dihexyl, methyl ethyl, ethyl propyl, ethyl isobutyl and pentamethylene sulphone; aliphatic, cycloaliphatic or aromatic hydrocarbons such as pentane, hexane, heptane, octane, nonane; for example white spirits with components having boiling points in the range, for example, from 40° C. to 250° C., cymene, benzine fractions within a boiling point range from 70° C. to 190° C., cyclohexane, methylcyclohexane, petroleum ether, ligroin, octane, benzene, toluene, chlorobenzene, bromobenzene, nitrobenzene, xylene; esters such as methyl, ethyl, butyl and isobutyl acetate, and also dimethyl, dibutyl and ethylene carbonate; amides such as hexamethylenephosphoramide, formamide, N-methylformamide, N,N-dimethylformamide, N,N-dipropylformamide, N,N-dibutylformamide, N-methylpyrrolidone, N-methylcaprolactam, 1,3-dimethyl-3,4,5,6-tetrahydro-2(1H)-pyrimidine, octylpyrrolidone, octylcaprolactam, 1,3-dimethyl-2-imidazolinedione, N-formylpiperidine, N,N′-1,4-diformylpiperazine; ketones such as acetone, acetophenone, methyl ethyl ketone, methyl butyl ketone.

Preferred cosolvents are: methanol, ethanol, THF, n-butanol.

In a particularly preferred embodiment, the reaction proceeds in a mixture of water and n-butanol.

The catalysts used may be phase transfer catalysts, for example quaternary ammonium salts.

In addition, the process according to the invention may proceed in aqueous biphasic systems. In this case, a further solvent of zero or only very limited water miscibility is used.

In step (i), the cysteamine hydrochloride or the cysteamine is dissolved in a solution of alkali metal hydroxides, alkali metal carbonates or alkali metal hydrogencarbonates. The solvent used may be water or a mixture with water. This operation can be effected at room temperature. Subsequently, the solution is cooled to a temperature of 5 to 40° C., preferably to 10 to 20° C. The dimethyl N-cyanocarbonimidodithiocarbonate is metered in (optionally as a melt).

The molar ratio of cysteamine hydrochloride to dimethyl N-cyanocarbonimidodithiocarbonate may be from 1:0.7 to 1:1.5, preferably 1:0.95 to 1:1.05.

After the metered addition has ended, the mixture is stirred at temperatures of 10 to 25° C. for another 0.5 to 10 hours, preferably 1 to 4 hours. Longer reaction times are uncritical.

The reaction is appropriately performed under atmospheric pressure, but it is also possible to work under reduced or elevated pressure.

In step (ii), further base can be added in the form of alkali metal hydroxides, alkali metal carbonates or alkali metal hydrogencarbonates. 2-Chloro-5-chloromethylpyridine is added in dissolved form, for example in n-butanol, or as a melt. The process in step (ii) can be performed within a wide temperature range, for example between 30° C. and 100° C., preferably between 50° C. and 80° C.

The reaction is appropriately performed under atmospheric pressure, but it is also possible to work under reduced or elevated pressure.

In the practical performance of the process, for example, 1 mol of compound (I) is reacted with 1 mol of the formula (II) and with 1.05 mol of the formula (III).

The process according to the invention can be performed batchwise or continuously. The process can also be performed under standard pressure, reduced pressure or elevated pressure.

The workup can be effected by filtration or extraction.

The process according to the invention for preparing [3-[(6-chloro-3-pyridinyl)methyl]-2-thiazolidinylidene]cyanamide is described in the examples which follow, which further illustrate the above description. However, the examples should not be interpreted in a restrictive manner.

### PREPARATION EXAMPLES

### Example 1

13.7 g of sodium carbonate (0.129 mol) are dissolved in 78 g of water. At RT, 15.0 g of 98% cysteamine hydrochloride (0.129 mol) are added. The suspension is cooled to 10° C. and admixed in portions with 21.0 g of 90% dimethyl cyanimidodithiocarbonate (0.129 mol). Gentle evolution of gas is immediately evident. The mixture is then allowed to come to the temperature of 20° C. within 2 h. HPLC monitoring shows complete conversion.

130 ml of n-butanol are added. The suspension is freed of the water on a water separator under reduced pressure at max. 50° C. Subsequently, the suspension, after ventilation, is admixed with 39.3 g of potassium carbonate (0.285 mol) and heated to 75° C. The mixture is stirred for a further 1 h.

Within 2 h, a solution of 22.1 g (0.136 mol) of 2-chloro-5-chloromethylpyridine in 44.1 g of n-butanol is added dropwise. The mixture is then heated to 80° C. and stirred at this temperature for 2 h. After cooling to 70° C., 130 ml of water at 70° C. are added and the phases are separated at 70° C. The aqueous phase is extracted twice at 70° C. with 20 ml each time of n-butanol. The combined butanol phases are cooled to 25° C. The precipitated solid is filtered off with suction. The mother liquor was cooled to −5° C. The precipitated solid is filtered off with suction through the first solid and washed once with 20 ml of n-butanol at −10° C. The resulting solid is dried in a vacuum drying cabinet at 40° C.

This gives 26.5 g of white solid with a purity of 98.2% (corresponds to 79.6% of theory).

¹H NMR, DMSO, 298 K: 3.50 ppm (t) 2H; 3.91 ppm (t) 2H; 4.64 ppm (s) 2H; 7.54 ppm (d) 1H; 7.79 ppm (dd) 1H; 8.38 ppm (d) 1H.

1. Process for preparing [3-[(6-chloro-3-pyridinyl)methyl]-2-thiazolidinylidene]cyanamide, comprising:
(i) reacting dimethyl N-cyanocarbonimidodithiocarbonate and cysteamine and/or a salt thereof in the presence of a base to form a reaction mixture;
(ii) reacting the reaction mixture with 5-chloromethyl-2-chloropyridine.

2. Process according to claim 1, in which the cysteamine is used as the salt of formula (I) 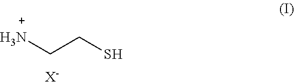
and X⁻ is selected from the group consisting of chloride, sulphate and hydrogensulphate.

3. Process according to claim 1, in the presence of at least one of the following bases: sodium hydroxide, potassium hydroxide, sodium hydrogencarbonate, potassium hydrogencarbonate, sodium carbonate and potassium carbonate.

4. Process according to claim 1, performed in a mixture of water and n-butanol.

5. Process according to claim 2, in the presence of at least_one of the following bases: sodium hydroxide, potassium hydroxide, sodium hydrogencarbonate, potassium hydrogencarbonate, sodium carbonate and potassium carbonate.

6. Process according to claim 2, performed in a mixture of water and n-butanol.

7. Process according to claim 3, performed in a mixture of water and n-butanol.
